(19) United States
(12) Patent Application Publication
(10) Pub. No.: US 2020/0129774 A1
(43) Pub. Date: Apr. 30, 2020

(54) MAGNET ASSEMBLY AND PACKAGING

(71) Applicant: Cardiac Pacemakers, Inc., St. Paul, MN (US)

(72) Inventors: David B. Joba, Minneapolis, MN (US); David P. Stieper, North Branch, MN (US); Alicia R. Brzezinski, Big Lake, MN (US); Bruce Fullerton, Hudson, WI (US)

(21) Appl. No.: 16/665,216

(22) Filed: Oct. 28, 2019

Related U.S. Application Data
(60) Provisional application No. 62/751,679, filed on Oct. 28, 2018.

(51) Int. Cl. A61N 1/375; A61N 1/39; A61B 5/11; A61B 7/04; A61B 5/024; A61B 5/0215; A61B 5/00; A61B 5/053

(52) U.S. Cl. CPC A61N 1/37512; A61N 1/3956; A61B 5/1118; A61B 7/04; A61B 5/02444; A61B 2562/162; A61B 5/4866; A61B 5/0538; A61B 5/6847; A61B 2562/0219; A61B 2562/164; A61B 5/0215

(57) ABSTRACT

A magnet assembly is configured to facilitate causing a state change of an implantable medical device (IMD), the IMD having a magnetic field sensor, and the magnet assembly including a disc-shaped magnet; and a magnet housing at least partially encasing the magnet. A medical device kit includes the magnet assembly and a packaging assembly including a box having a lid and a foam insert configured to be placed within the box, the foam insert including a slot defined therein for receiving the magnet assembly.

### CROSS-REFERENCE TO RELATED APPLICATION

This application claims priority to Provisional Application No. 62/751,679, filed Oct. 28, 2019, which is herein incorporated by reference in its entirety.

### TECHNICAL FIELD

The present disclosure relates to magnet assemblies configured to interact with implantable medical devices. More specifically, the disclosure relates to magnet assemblies having magnets disposed within housings.

### BACKGROUND

Implantable medical devices (IMDs) may be configured to communicate with external devices such as, for example, mobile devices (e.g., smartphones, tablet computers, etc.) to facilitate recording of data, symptom tracking, and/or any number of other cooperative functions. This communication may be performed using a radio frequency communication technology such as, for example, Bluetooth. These types of communication technologies may reduce the lifetime of the IMD by draining power sources, putting stress on circuits, and/or the like.

### SUMMARY

In an Example 1, a magnet assembly configured to facilitate causing a state change of an implantable medical device (IMD), the IMD having a magnetic field sensor, the magnet assembly comprising: a disc-shaped magnet; and a magnet housing at least partially encasing the magnet.

In an Example 2, the magnet assembly of Example 1, the magnet housing comprising a generally rectangular shape.

In an Example 3, the magnet assembly of Example 2, wherein the magnet housing includes a length and a width, wherein the length is equal to the width so that the magnet housing has a generally square shape.

In an Example 4, the magnet assembly of any of Examples 1-3, wherein the magnet housing comprises an upper wall having an outer surface and an opposite inner surface, a lower wall having an outer surface and an opposite inner surface, and a side wall having an outer surface and an opposite inner surface.

In an Example 5, the magnet assembly of Example 4, wherein the side wall of the magnet housing is tapered such that a length and/or width of the housing increases toward the lower wall.

In an Example 6, the magnet assembly of either of Examples 4 or 5, wherein the outer surface of the upper wall is approximately flat, thereby facilitating intuitive orientation of the magnet with respect to the IMD.

In an Example 7, the magnet assembly of any of Examples 4-6, wherein the outer surface of the lower wall is approximately flat to facilitate coupling the magnet housing to an external device.

In an Example 8, the magnet assembly of Example 7, further comprising an adhesive configured to be disposed between the outer surface of the lower wall and an outer surface of the external device, wherein the adhesive is configured to couple the magnet housing to the external device.

In an Example 9, the magnet assembly of Example 8, further comprising one or more pry-off features configured to facilitate gripping the magnet housing and removing it from the outer surface of the external device.

In an Example 10, the magnet assembly of Example 9, wherein the one or more pry-off features comprise a notch defined in the lower wall of the magnet assembly.

In an Example 11, the magnet assembly of Example 9, the one or more pry-off features comprising at least two pry-off features, in opposite corners of the magnet housing, at the ends of the longest dimension of the magnet housing.

In an Example 12, the magnet assembly of any of Examples 1-11, further comprising an aperture defined through the magnet housing.

In an Example 13, a medical device kit, comprising: a magnet assembly, the magnet assembly comprising a disc-shaped magnet and a magnet housing at least partially encasing the disc-shaped magnet; and a packaging assembly comprising a box having a lid and a foam insert configured to be placed within the box, the foam insert comprising a slot defined therein for receiving the magnet assembly.

In an Example 14, the medical device kit of Example 13, wherein the foam insert has a generally rectangular shape, wherein a length of the foam insert is greater than a width of the foam insert.

In an Example 15, the medical device kit of Example 14, wherein the slot is configured to retain the magnet assembly in an orientation in which the direction of the magnetic field is approximately parallel to the length of the foam insert.

In an Example 16, a magnet assembly configured to facilitate causing a state change of an implantable medical device (IMD), the IMD having a magnetic field sensor, the magnet assembly comprising: a disc-shaped magnet; and a magnet housing at least partially encasing the magnet.

In an Example 17, the magnet assembly of Example 16, the magnet housing comprising a generally rectangular shape.

In an Example 18, the magnet assembly of Example 17, wherein the magnet housing includes a length and a width, wherein the length is equal to the width so that the magnet housing has a generally square shape.

In an Example 19, the magnet assembly of Example 16, wherein the magnet housing comprises an upper wall having an outer surface and an opposite inner surface, a lower wall having an outer surface and an opposite inner surface, and a side wall having an outer surface and an opposite inner surface.

In an Example 20, the magnet assembly of Example 19, wherein the side wall of the magnet housing is tapered such that a length and/or width of the housing increases toward the lower wall.

In an Example 21, the magnet assembly of Example 19, wherein the outer surface of the upper wall is approximately flat, thereby facilitating intuitive orientation of the magnet with respect to the IMD.

In an Example 22, the magnet assembly of Example 19, wherein the outer surface of the lower wall is approximately flat to facilitate coupling the magnet housing to an external device.

In an Example 23, the magnet assembly of Example 22, further comprising an adhesive configured to be disposed between the outer surface of the lower wall and an outer surface of the external device, wherein the adhesive is configured to couple the magnet housing to the external device.

In an Example 24, the magnet assembly of Example 23, further comprising one or more pry-off features configured to facilitate gripping the magnet housing and removing it from the outer surface of the external device.

In an Example 25, the magnet assembly of Example 24, wherein the one or more pry-off features comprise a notch defined in the lower wall of the magnet assembly.

In an Example 26, the magnet assembly of Example 24, the one or more pry-off features comprising at least two pry-off features, in opposite corners of the magnet housing, at the ends of the longest dimension of the magnet housing.

In an Example 27, the magnet assembly of Example 16, further comprising an aperture defined through the magnet housing.

In an Example 28, a magnet assembly configured to facilitate causing a state change of an implantable medical device (IMD), the IMD having a magnetic field sensor, the magnet assembly comprising: a disc-shaped magnet; and a magnet housing at least partially encasing the magnet, wherein the magnet housing comprises an upper wall having an outer surface and an opposite inner surface, a lower wall having an outer surface and an opposite inner surface, and a side wall having an outer surface and an opposite inner surface, and wherein the side wall of the magnet housing is tapered such that a length and/or width of the housing increases toward the lower wall.

In an Example 29, the magnet assembly of Example 28, further comprising an adhesive configured to be disposed between the outer surface of the lower wall and an outer surface of the external device, wherein the adhesive is configured to couple the magnet housing to the external device.

In an Example 30, the magnet assembly of Example 29, further comprising one or more pry-off features configured to facilitate gripping the magnet housing and removing it from the outer surface of the external device.

In an Example 31, the magnet assembly of Example 30, the one or more pry-off features comprising at least two pry-off features, in opposite corners of the magnet housing, at the ends of the longest dimension of the magnet housing.

In an Example 32, the magnet assembly of Example 28, further comprising an aperture defined through the magnet housing.

In an Example 33, a medical device kit, comprising: a magnet assembly, the magnet assembly comprising a disc-shaped magnet and a magnet housing at least partially encasing the disc-shaped magnet; and a packaging assembly comprising a box having a lid and a foam insert configured to be placed within the box, the foam insert comprising a slot defined therein for receiving the magnet assembly.

In an Example 34, the medical device kit of Example 33, wherein the foam insert has a generally rectangular shape, wherein a length of the foam insert is greater than a width of the foam insert.

In an Example 35, the medical device kit of Example 34, wherein the slot is configured to retain the magnet assembly in an orientation in which the direction of the magnetic field is approximately parallel to the length of the foam insert.

While multiple embodiments are disclosed, still other embodiments of the presently disclosed subject matter will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic illustration of a medical system including an implantable medical device (IMD) implanted in a subject and an external device (ED) configured to communicate with the IMD, in accordance with embodiments of the disclosure.

FIG. 2A is a perspective view of an illustrative magnet, in accordance with embodiments of the subject matter disclosed herein.

FIG. 2B is a schematic diagram depicting an illustrative magnetic field provided by the magnet depicted in FIG. 2A, in accordance with embodiments of the subject matter disclosed herein.

FIG. 3A is a top perspective view of an illustrative magnet assembly, in accordance with embodiments of the subject matter disclosed herein.

FIG. 3B is a partially cut-away perspective view of the illustrative magnet assembly depicted in FIG. 3A, in accordance with embodiments of the subject matter disclosed herein.

FIG. 3C is a close-up perspective view of a portion of the magnet assembly depicted in FIGS. 3A and 3B, in accordance with embodiments of the subject matter disclosed herein.

FIG. 3D is a side view of the magnet assembly depicted in FIGS. 3A-3C, in accordance with embodiments of the subject matter disclosed herein.

FIG. 4A is a top perspective view of another illustrative magnet assembly, in accordance with embodiments of the subject matter disclosed herein.

FIG. 4B is a partially cut-away perspective view of the illustrative magnet assembly depicted in FIG. 4A, in accordance with embodiments of the subject matter disclosed herein.

FIG. 4C is a perspective view of the magnet assembly depicted in FIGS. 4A and 4B, in accordance with embodiments of the subject matter disclosed herein.

FIG. 4D is a side view of the magnet assembly depicted in FIGS. 4A-4C, in accordance with embodiments of the subject matter disclosed herein.

FIG. 5A is a lower perspective view of an illustrative magnet assembly, in accordance with embodiments of the subject matter disclosed herein.

FIG. 5B is a perspective view of the illustrative magnet assembly depicted in FIG. 5A coupled to an illustrative mobile device, in accordance with embodiments of the subject matter disclosed herein.

FIG. 6A is an exploded perspective view of an illustrative packaging assembly, in accordance with embodiments of the subject matter disclosed herein.

FIG. 6B is a cross-sectional perspective view of the illustrative packaging assembly depicted in FIG. 6A, in accordance with embodiments of the subject matter disclosed herein.

While the disclosed subject matter is amenable to various modifications and alternative forms, specific embodiments have been shown by way of example in the drawings and are described in detail below. The intention, however, is not to limit the subject matter disclosed herein to the particular embodiments described. On the contrary, the disclosure is intended to cover all modifications, equivalents, and alternatives falling within the scope of the subject matter disclosed herein, and as defined by the appended claims.

As used herein in association with values (e.g., terms of magnitude, measurement, and/or other degrees of qualitative and/or quantitative observations that are used herein with respect to characteristics (e.g., dimensions, measurements, attributes, components, etc.) and/or ranges thereof, of tangible things (e.g., products, inventory, etc.) and/or intangible things (e.g., data, electronic representations of currency, accounts, information, portions of things (e.g., percentages, fractions), calculations, data models, dynamic system models, algorithms, parameters, etc.), “about” and “approximately” may be used, interchangeably, to refer to a value, configuration, orientation, and/or other characteristic that is equal to (or the same as) the stated value, configuration, orientation, and/or other characteristic or equal to (or the same as) a value, configuration, orientation, and/or other characteristic that is reasonably close to the stated value, configuration, orientation, and/or other characteristic, but that may differ by a reasonably small amount such as will be understood, and readily ascertained, by individuals having ordinary skill in the relevant arts to be attributable to measurement error; differences in measurement and/or manufacturing equipment calibration; human error in reading and/or setting measurements; adjustments made to optimize performance and/or structural parameters in view of other measurements (e.g., measurements associated with other things); particular implementation scenarios; imprecise adjustment and/or manipulation of things, settings, and/or measurements by a person, a computing device, and/or a machine; system tolerances; control loops; machine-learning; foreseeable variations (e.g., statistically insignificant variations, chaotic variations, system and/or model instabilities, etc.); preferences; and/or the like.

The terms “up,” “upper,” and “upward,” and variations thereof, are used throughout this disclosure for the sole purpose of clarity of description and are only intended to refer to a relative direction (i.e., a certain direction that is to be distinguished from another direction), and are not meant to be interpreted to mean an absolute direction. Similarly, the terms “down,” “lower,” and “downward,” and variations thereof, are used throughout this disclosure for the sole purpose of clarity of description and are only intended to refer to a relative direction that is at least approximately opposite a direction referred to by one or more of the terms “up,” “upper,” and “upward,” and variations thereof.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

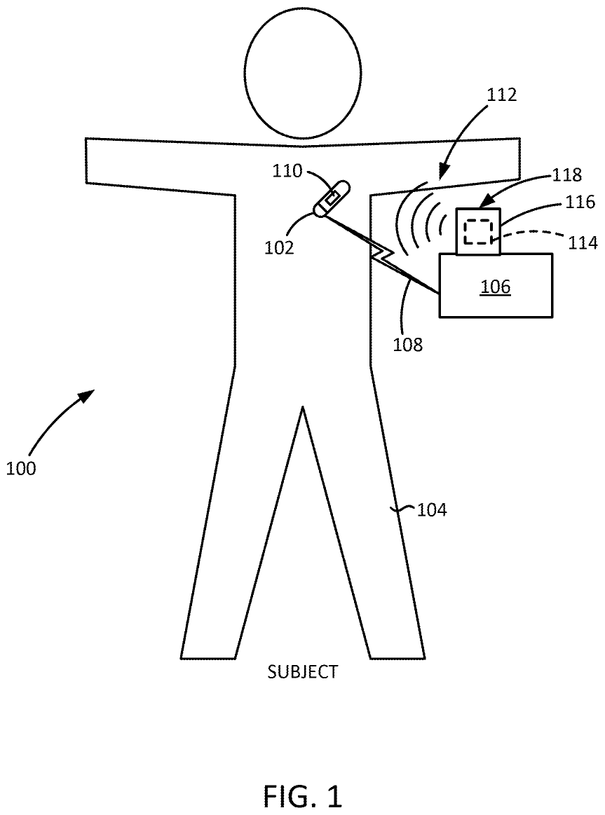

FIG. 1 is a schematic illustration of a medical system 100 including an implantable medical device (IMD) 102 implanted in a subject 104 and an external device (ED) 106 configured to communicate with the IMD 102, in accordance with embodiments of the disclosure. According to embodiments, the system 100 may include any number of other devices. The subject 104 may be a human, a dog, a pig, and/or any other animal having physiological parameters that can be recorded. For example, in embodiments, the subject 104 may be a human patient.

In embodiments, the IMD 102 and the ED 106 may be communicatively coupled via a communication link 108. In embodiments, the communication link 108 may be, or include, a wired link (e.g., a link accomplished via a physical connection) and/or a non-wired communication link such as, for example, a short-range radio link, such as Bluetooth, Bluetooth Low Energy, IEEE 802.11, near-field communication (NFC), WiFi, a proprietary wireless protocol, and/or the like. The communication link 108 may be, include, or be included in any number of different types of communication networks such as, for example, a bus network, a short messaging service (SMS), a local area network (LAN), a wireless LAN (WLAN), a wide area network (WAN), the Internet, a P2P network, custom-designed communication or messaging protocols, and/or the like. The communication link 108 may include a combination of multiple networks. The term “communication link” may refer to an ability to communicate some type of information in at least one direction between at least two devices, and should not be understood to be limited to a direct, persistent, or otherwise limited communication channel. That is, according to embodiments, the communication link 108 may be a persistent communication link, an intermittent communication link, an ad-hoc communication link, and/or the like. The communication link 108 may refer to direct communications between the IMD 102 and the ED 106 and/or indirect communications that travel between the IMD 102 and the ED 106 via at least one other device (e.g., a repeater, router, hub, and/or the like). The communication link 108 may facilitate uni-directional and/or bi-directional communication between multilayer IMD 102 and the ED 106. Data and/or control signals may be transmitted between the IMD 102 and the ED 106. In embodiments, subject data may be downloaded from the IMD 102 and/r the ED 106 periodically or on command. The clinician and/or the subject 104 may communicate with the IMD 102 and/or the ED 106, for example, to acquire subject data or to initiate, terminate and/or modify recording and/or therapy. In embodiments, the communication link 108 may facilitate encryption and/or other methods to increase data transmission safety.

According to embodiments, the IMD 102 may include any type of IMD, any number of different components of an implantable system, and/or the like. For example, the IMD 102 may include a control device, a monitoring device, a pacemaker, an implantable cardioverter defibrillator (ICD), a cardiac resynchronization therapy (CRT) device and/or the like, and may be an implantable medical device known in the art or later developed, for providing therapy to, and/or diagnostic data about, the subject 104 and/or the IMD 102. In various embodiments, the IMD 102 may include both defibrillation and pacing/CRT capabilities (e.g., a CRT-D device).

In embodiments, the IMD 102 may be implanted subcutaneously within an implantation location or pocket in the patient's chest or abdomen and may be configured to monitor (e.g., sense and/or record) physiological parameters such as, for example, parameters associated with the patient's heart. In embodiments, the IMD 102 may be an implantable cardiac monitor (ICM) (e.g., an implantable diagnostic monitor (IDM), an implantable loop recorder (ILR), etc.) configured to record physiological parameters such as, for example, one or more cardiac electrical signals, heart sounds, heart rate, blood pressure measurements, oxygen saturations, and/or the like.

In embodiments, the IMD 102 may be configured to monitor physiological parameters that may include one or more signals indicative of a patient's physical activity level and/or metabolic level, such as an acceleration signal. In embodiments, the IMD 102 may be configured to sense intrathoracic impedance, from which various respiratory parameters may be derived, including, for example, respiratory tidal volume and minute ventilation. Sensors and associated circuitry may be incorporated in connection with the IMD 102 for detecting one or more body movement or body posture and/or position related signals. For example, accelerometers and/or GPS devices may be employed to detect patient activity, patient location, body orientation, and/or torso position. The IMD 102 may be configured to sense and/or record at regular intervals, continuously, and/or in response to a detected event.

In various embodiments, the ED 106 may be a device that is configured to be portable with the subject 104, e.g., by being integrated into a vest, belt, harness, sticker; placed into a pocket, a purse, or a backpack; carried in the subject's hand; adhered to the subject's skin; and/or the like, or otherwise operatively (and/or physically) coupled to the subject 104. According to embodiments, the ED 106 may be a mobile device such as, for example, a cellphone, a smartphone, a personal digital assistant (PDA), a tablet computer, and/or the like. The ED 106 may be configured to monitor (e.g., sense and/or record) physiological parameters associated with the subject 104 and/or provide therapy to the subject 104. For example, the ED 106 may be, or include, a wearable cardiac defibrillator (WCD) such as a vest that includes one or more defibrillation electrodes. In embodiments, the ED 106 may include any number of different therapy components such as, for example, a defibrillation component, a drug delivery component, a neurostimulation component, a neuromodulation component, a temperature regulation component, and/or the like. In embodiments, the ED 106 may include limited functionality, e.g., defibrillation shock delivery and communication capabilities, with arrhythmia detection, classification and/or therapy command/control being performed by a separate device such as, for example, the IMD 102.

In embodiments, the ED 106 may include sensing components such as, for example, one or more surface electrodes configured to obtain an electrocardiogram (ECG), one or more accelerometers configured to detect motion associated with the patient 104, one or more respiratory sensors configured to obtain respiration information, one or more environmental sensors configured to obtain information about the external environment (e.g., temperature, air quality, humidity, carbon monoxide level, oxygen level, barometric pressure, light intensity, sound, and/or the like) surrounding the patient 104, and/or the like. In embodiments, the ED 106 may be configured to measure parameters relating to the human body, such as temperature (e.g., a thermometer), blood pressure (e.g., a sphygmomanometer), blood characteristics (e.g., glucose levels), body weight, physical strength, mental acuity, diet, heart characteristics, relative geographic position (e.g., a Global Positioning System (GPS)), and/or the like.

According to embodiments, the IMD 102 is configured to communicate with the ED 106 to facilitate monitoring, therapy, and/or other medical services. For example, the IMD 102 may be configured to provide data to the ED 106 so that the ED 106 can store the data, process the data, confirm diagnoses determined by the IMD 102, and/or the like. The ED 106 may be configured to provide data to the IMD 102, provide control instructions to the IMD 102, and/or the like. In embodiments, the communication between the IMD 102 and ED 106, as indicated above, may be facilitated using a radio frequency communication technology such as Bluetooth, which may be referred to herein as a “primary” communication technology. To facilitate longevity of the IMD 102 and/or ED 106, a secondary communication technology may be used to facilitate initiating communication between the IMD 102 and the ED 106. This secondary communication technology may be configured to consume less power than the primary communication technology, thereby facilitating longevity of the power source of the IMD 102 and/or the ED 106, the circuitry of the IMD 102 and/or the ED 106, and/or the like.

According to embodiments, the secondary communication technology may be, or include, magnetic interactions. That is, for example, the IMD 102 may include a magnetic sensor 110 configured to detect and/or otherwise react to a magnetic field 112 provided by a magnet 114. In this manner, the magnet 114 may be placed into the proximity of the magnetic sensor 110, which detects the magnetic field 112 provided by the magnet 114. In response to detecting the magnetic field 112, the magnetic sensor 110 may cause the IMD 102 change one or more states. That is, for example, the magnetic sensor 110 may be configured to cause, in response to detecting the magnetic field 112, a communication module of the IMD 102 to activate (e.g., to cause the IMD 102 to begin listening for a communication from the ED 106), a sensing module of the IMD 102 to perform a sensing action, a therapy module of the IMD 102 to perform a therapy action, and/or the like.

In embodiments, the magnetic sensor 110 may be, or include, any number of different types of magnetometers such as, for example, Hall effect sensors, magnetoresistive devices, microelectromechanical systems (MEMS) magnetometers, and/or the like. The magnet 114 may be, or include, any kind of disc magnet configured to produce a magnetic field detectable by the magnetic sensor 110. In embodiments, the magnet 114 may be made of any number of different types of materials including, for example, neodymium magnets such as Neodymium-Iron-Boron magnets. According to embodiments, the magnet 114 may be encased in a magnet housing 116 to form a magnet assembly 118. The magnet assembly may be, in embodiments, independent of the ED 106, coupled to the ED 106, and/or integrated with the ED 106.

The illustrative medical system 100 shown in FIG. 1 is not intended to suggest any limitation as to the scope of use or functionality of embodiments of the present disclosure. The illustrative system 100 should not be interpreted as having any dependency or requirement related to any single component or combination of components illustrated therein. Additionally, various components depicted in FIG. 1 may be, in embodiments, integrated with various ones of the other components depicted therein (and/or components not illustrated), all of which are considered to be within the ambit of the subject matter disclosed herein.

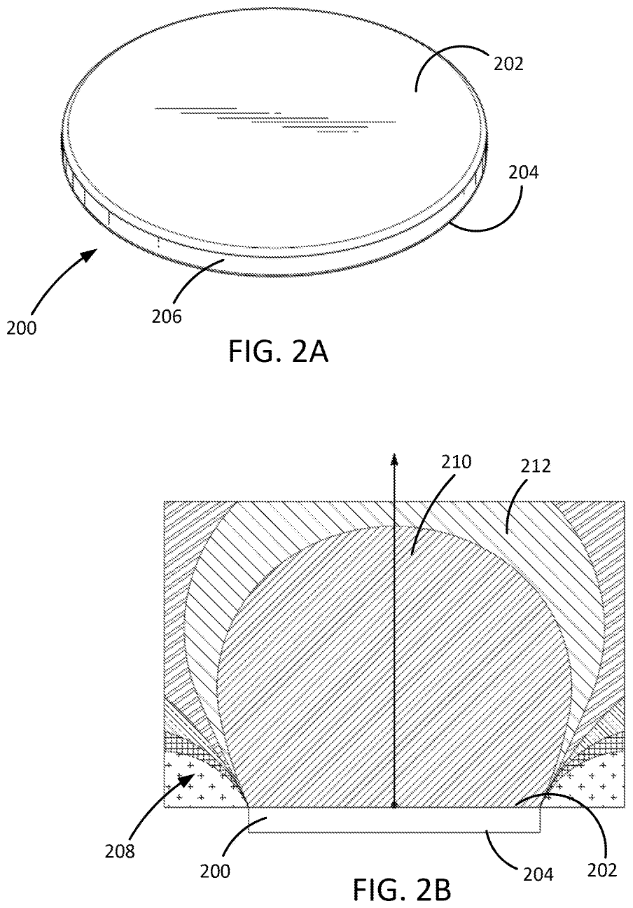

FIG. 2A is a perspective view of an illustrative magnet 200, in accordance with embodiments of the subject matter disclosed herein. The magnet 200 may be, or be similar to, the magnet 114 depicted in FIG. 1. As shown, the magnet 200 is a disc-shaped magnet that includes a first face 202, an opposite and parallel second face 204, and an edge 206 extending between the two faces 202 and 204. According to embodiments, the magnet 200 may have a diameter of between approximately three-quarters of an inch and approximately 2 inches, and may have a thickness of between approximately one twelfth of an inch and approximately one half of an inch. For example, in embodiments, the magnet 200 may have a diameter of approximately one and one-half inches and a thickness of approximately one eighth of an inch.

The magnet 200 may be configured to provide a magnetic field that is directed axially such that, at the face 202 and/or 204, the magnetic field is approximately perpendicular to the surface of the face 202 and/or 204. FIG. 2B is a schematic diagram depicting an illustrative magnetic field 208 provided by the magnet 200, in accordance with embodiments of the subject matter disclosed herein. As shown, at least a component of the magnetic field 208 (e.g., the most relevant component of the field for use in interacting with an IMD) extends away from the face 202 in a symmetrical manner, producing field regions 210 and 212 of relatively uniformly decreasing field strength. According to embodiments, this type of symmetric magnetic field 208 may facilitate allowing the magnet 200 to be used in a number of different orientations. In this manner, for example, a subject (e.g., the subject 104 depicted in FIG. 1) may be able to use the magnet 200 to cause a state change to an IMD (e.g., the IMD 102 depicted in FIG. 1) without having to use a precise placement or orientation of the magnet 200.

Magnets such as, for example, the disc magnet 200 depicted in FIGS. 2A and 2B may have a tendency to corrode when exposed to the environment and, in particular, when exposed to oils and contaminants present on a subject's skin. Additionally, in embodiments, the magnet may include nickel plating, to which a percentage of the population is allergic. Accordingly, to provide protection from corrosion and other adverse effects to the magnet and/or the user of the magnet (e.g., the subject 104 depicted in FIG. 1), a magnet housing may be used to encase, or at least partially encase, the magnet, in accordance with embodiments of the disclosure.

The illustrative magnet 200 shown in FIGS. 2A and 2B is not intended to suggest any limitation as to the scope of use or functionality of embodiments of the present disclosure. The illustrative magnet 200 should not be interpreted as having any dependency or requirement related to any single component or combination of components illustrated therein. Additionally, various components depicted in FIGS. 2A and 2B may be, in embodiments, integrated with various ones of the other components depicted therein (and/or components not illustrated), all of which are considered to be within the ambit of the subject matter disclosed herein.

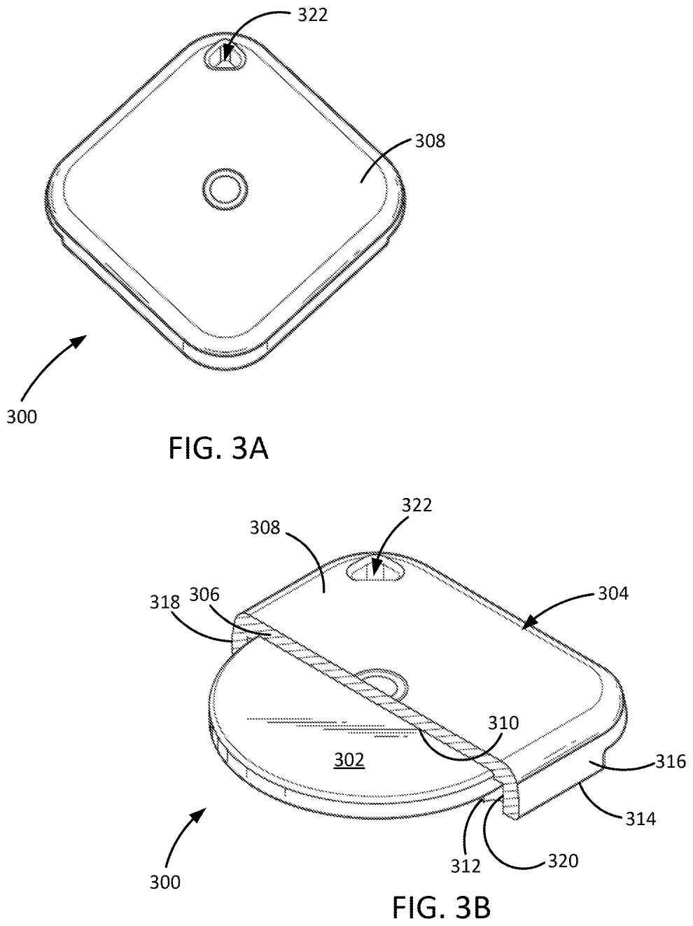

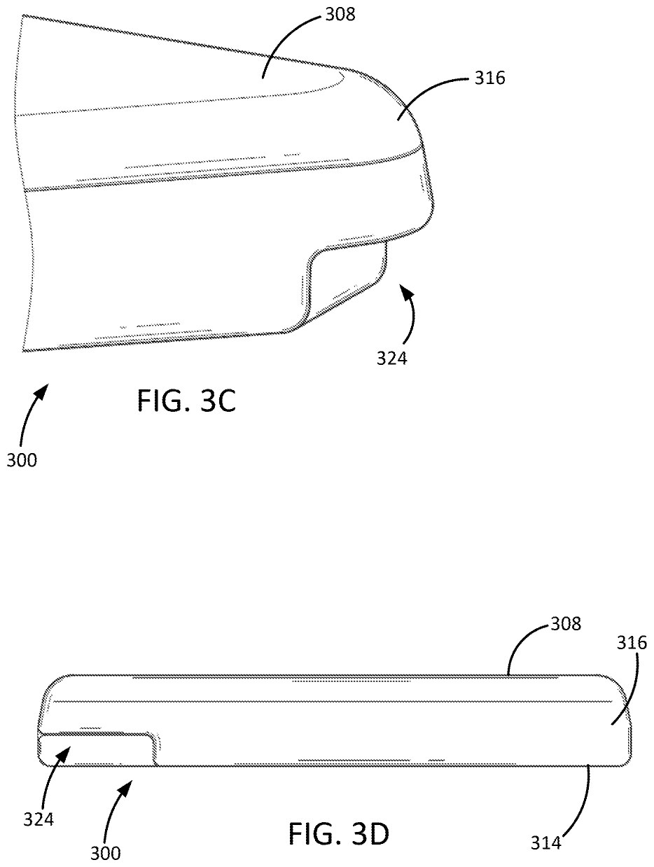

FIG. 3A is a top perspective view of an illustrative magnet assembly 300, in accordance with embodiments of the subject matter disclosed herein; and FIG. 3B is a partially cut-away perspective view of the illustrative magnet assembly 300 depicted in FIG. 3A, in accordance with embodiments of the subject matter disclosed herein. As shown in FIGS. 3A and 3B, the magnet assembly 300 includes a magnet 302 disposed within a magnet housing 304. The magnet 302 may be, or be similar to, the magnet 114 depicted in FIG. 1 and/or the magnet 200 depicted in FIGS. 2A and 2B. The magnet housing 304 may be, or be similar to, the magnet housing 116 depicted in FIG. 1, and may be configured to encase, or at least partially encase, the magnet 302. That is, for example, the magnet housing 304 may be configured such that any holes therein that expose the magnet 302 are small enough to prevent human fingers from passing through the holes and touching the magnet 302.

The magnet housing 304 may be made of any number of different materials configured to encase (or at least partially encase) the magnet without interfering with the magnetic field produced by the magnet so much as to prevent detection of the magnetic field by a magnet sensor (e.g., the magnet sensor 110 depicted in FIG. 1). For example, the magnet housing may be made of a plastic material, a polymer material, and/or the like. For example, the magnet housing may be made of any number of various thermoplastics such as acrylonitrile butadiene styrene (ABS) a blend of polycarbonate and acrylonitrile butadiene styrene (PC/ABS), thermoplastic elastomers such as polyether block amide (PEBA), thermoplastic polyurethanes, and/or the like. According to embodiments, the magnet assembly 300 may be produced using any number of different manufacturing processes. For example, in embodiments, the magnet assembly 300 may be formed using a one-shot insert mold with the magnet 302 in the housing 304. When the magnet assembly 300 is formed, the magnet 302 may be a molding blank disc that may be magnetized after formation of the magnet assembly 300. In embodiments, the magnet 302 may be approximately centered within the housing 304.

As is further shown in FIGS. 3A and 3B, the magnet housing 304 includes an upper wall 306 having an outer surface 308 and an opposite inner surface 310, a lower wall 312 having an outer surface 314 and an opposite inner surface (not shown), and a side wall 316 having an outer surface 318 and an opposite inner surface 320. According to embodiments, the side wall 316 of the magnet housing may be tapered such that a length and/or width of the housing 302 increases toward the lower wall 312. This tapered side wall 316 may be configured to facilitate reducing shear loads on the magnet housing 302 when installed on a mobile device. An aperture 322 may be defined through the magnet housing 304, extending from the outer surface 308 of the upper wall 306 to the outer surface 314 of the lower wall 312. According to embodiments, the aperture 322 may be configured to receive a mechanism for holding the magnet assembly 300 such as, for example, a lanyard, a string, a clip, a keychain, and/or the like.

According to embodiments, the outer surfaces 308 and 314 of the upper and/or lower walls 306 and 312, respectively, may be configured to be approximately flat. For example, in embodiments, the outer surface 308 of the upper wall 306 may be approximately flat, which may, in embodiments, facilitate intuitive orientation of the magnet with respect to the IMD. The flat surface(s) also may facilitate coupling the magnet housing 304 to another object. The magnet assembly 300 may include an adhesive (not shown) such as, for example, an adhesive glue, tape, sticker, and/or the like, that is configured to be disposed on an outer surface 308 or 314 such that the magnet housing 304 may be coupled to an object by pressing the surface 308 or 314 with the adhesive against the object to which the magnet assembly 300 is to be coupled. That is, for example, the magnet housing 304 may be configured to be coupled to a mobile device such as, for example, the external device 106 depicted in FIG. 1. in embodiments, for example, the magnet assembly 300 may be coupled to a smartphone or other mobile device by affixing an adhesive to the magnet assembly such as, for example, a round adhesive having a diameter of between approximately one half of an inch to approximately two inches (e.g., one and one-half inches).

In embodiments, the magnet housing 304 may be configured to have a low profile (e.g., a thin, discreet appearance), which may facilitate, for example, coupling the magnet housing 304 to a mobile device such as a smartphone. That is, for example, the magnet housing 304 may have a length and/or a width of between approximately one-half of an inch and approximately two and one-half inches, and a thickness of between approximately one eighth of an inch and three quarters of an inch. That is, for example, in embodiments, the magnet housing 304 may have a length and/or a width of approximately 1.85 inches and a thickness of approximately one quarter of an inch. According to embodiments, the length and width may be configured to be between approximately 5 times and approximately ten times the thickness of the magnet 302. In embodiments, the magnet housing 404 may include a generally rectangular shape. In embodiments, the length and width may be configured to greater than approximately ten times the thickness of the magnet 302. In embodiments, the length and width of the magnet housing 304 may be approximately equal, so that the magnet housing 304 has a generally square shape, which the inventors have discovered has the best drop test results.

FIG. 3C is a close-up perspective view of a portion of the magnet assembly 300 depicted in FIGS. 3A and 3B, in accordance with embodiments of the subject matter disclosed herein; and FIG. 3D is a side view of the magnet assembly 300 depicted in FIGS. 3A-3C, in accordance with embodiments of the subject matter disclosed herein. As shown in FIGS. 3C and 3D, the magnet housing 304 may include one or more pry-off features 324 configured to facilitate gripping the magnet housing 304 and removing it from a surface to which it is adhered. As shown, a pry-off feature 324 may include, for example, a notch defined in the lower wall 306 and/or side wall 316. In embodiments, the magnet housing 302 may include one pry-off feature 324, two pry-off features 324, three pry-off features 324, and/or the like. For example, in embodiments, the magnet housing 302 may include two pry-off features 324, in opposite corners (or on opposite sides) of the housing 302, at the ends of the longest dimension of the magnet housing 302, thereby increasing the amount of torque that a user can apply to the housing 302 to remove it from the surface to which it is adhered.

The illustrative magnet assembly 300 shown in FIGS. 3A-3D is not intended to suggest any limitation as to the scope of use or functionality of embodiments of the present disclosure. The illustrative magnet assembly 300 should not be interpreted as having any dependency or requirement related to any single component or combination of components illustrated therein. Additionally, various components depicted in FIGS. 3A-3D may be, in embodiments, integrated with various ones of the other components depicted therein (and/or components not illustrated), all of which are considered to be within the ambit of the subject matter disclosed herein.

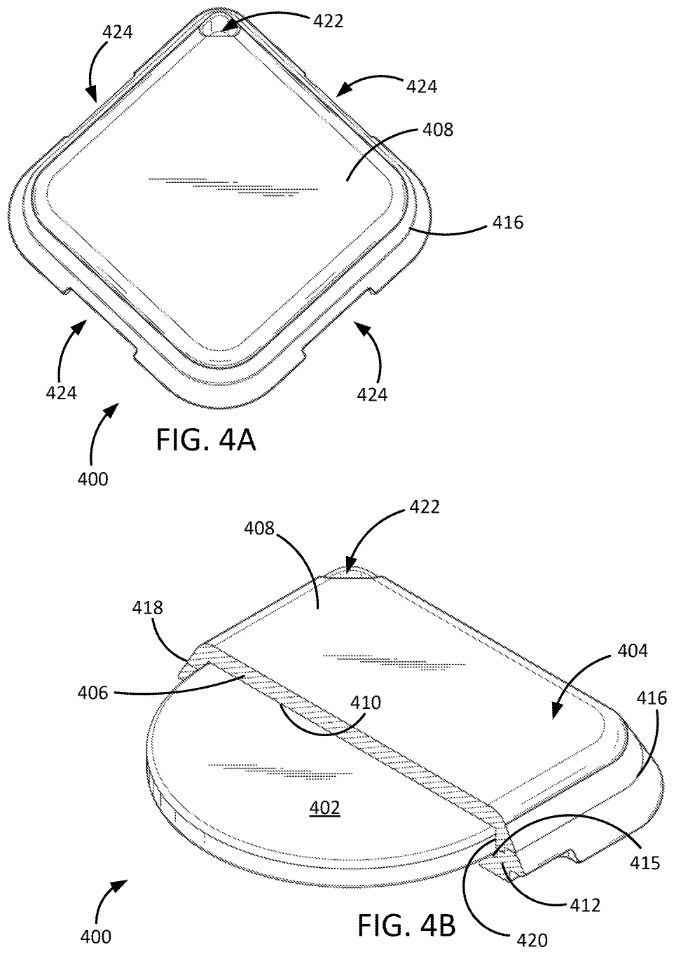

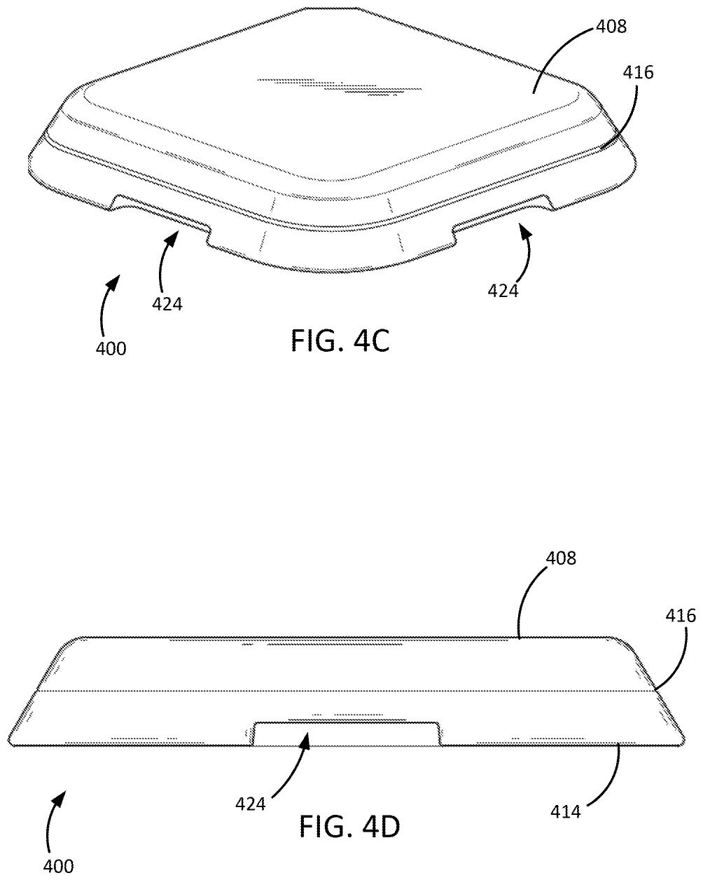

FIG. 4A is a top perspective view of another illustrative magnet assembly 400, in accordance with embodiments of the subject matter disclosed herein; and FIG. 4B is a partially cut-away perspective view of the illustrative magnet assembly 400 depicted in FIG. 4A, in accordance with embodiments of the subject matter disclosed herein. As shown in FIGS. 4A and 4B, the magnet assembly 400 includes a magnet 402 disposed within a magnet housing 404. The magnet 402 may be, or be similar to, the magnet 114 depicted in FIG. 1 and/or the magnet 200 depicted in FIGS. 2A and 2B. The magnet housing 404 may be, or be similar to, the magnet housing 116 depicted in FIG. 1, and may be configured to encase, or at least partially encase, the magnet 402. That is, for example, the magnet housing 404 may be configured such that any holes therein that expose the magnet 402 are small enough to prevent human fingers from passing through the holes and touching the magnet 402. The magnet housing 404 may be made of any number of different materials such as those from which the magnet housing 304 depicted in FIGS. 3A-3D is made, and may be made using the same or similar manufacturing processes as the magnet housing 304.

As is further shown in FIGS. 4A and 4B, the magnet housing 404 includes an upper wall 406 having an outer surface 408 and an opposite inner surface 410, a lower wall 412 having an outer surface 414 and an opposite inner surface 415, and a side wall 416 having an outer surface 418 and an opposite inner surface 420. According to embodiments, the side wall 416 of the magnet housing may be tapered such that a length and/or width of the housing 402 increases toward the lower wall 412. In embodiments, the taper of the side wall 416 may be steeper and/or more linear than the taper of the side wall 316. That is, for example, the side wall 416 may be configured to taper approximately linearly toward the lower wall 414 at approximately a forty-five degree angle with respect to a normal direction to the outer surface 408 of the upper wall 406. The angle of the taper may be any other angle suitable to produce a taper that reduces shear loads on the magnet housing and may include angles ranging between approximately fifteen degrees and approximately fifty-five degrees.

This tapered side wall 416 may be configured to facilitate reducing shear loads on the magnet housing 402 when installed on a mobile device. An aperture 422 may be defined through the magnet housing 404, extending from the outer surface 408 of the upper wall 406 to the outer surface 414 of the lower wall 412. According to embodiments, the aperture 422 may be configured to receive a mechanism for holding the magnet assembly 400 such as, for example, a lanyard, a string, a clip, a keychain, and/or the like.

According to embodiments, the outer surfaces 408 and 414 of the upper and/or lower walls 406 and 412, respectively, may be configured to be approximately flat. For example, the outer surface 408 of the upper wall 406 may be approximately flat, which may, in embodiments, facilitate intuitive orientation of the magnet with respect to the IMD. The flat surface(s) also may facilitate coupling the magnet housing 404 to another object (e.g., where the outer surface 414 of the lower wall 412 is approximately flat to provide a continuous interface with an external device). The magnet assembly 400 may include an adhesive (not shown) such as, for example, an adhesive glue, tape, sticker, and/or the like, that is configured to be disposed on an outer surface 408 or 414 such that the magnet housing 404 may be coupled to an object by pressing the surface 408 or 414 with the adhesive against the object to which the magnet assembly 400 is to be coupled. That is, for example, the magnet housing 404 may be configured to be coupled to a mobile device such as, for example, the external device 106 depicted in FIG. 1. In embodiments, for example, the magnet assembly 400 may be coupled to a smartphone or other mobile device by affixing an adhesive to the magnet assembly such as, for example, a round adhesive having a diameter of between approximately one half of an inch to approximately two inches (e.g., one and one-half inches).

In embodiments, the magnet housing 404 may be configured to have a low profile (e.g., a thin, discreet appearance), which may facilitate, for example, coupling the magnet housing 404 to a mobile device such as a smartphone. That is, for example, the magnet housing 404 may have a length and/or a width of between approximately one-half of an inch and approximately two and one-half inches, and a thickness of between approximately one eighth of an inch and three quarters of an inch. That is, for example, in embodiments, the magnet housing 404 may have a length and/or a width of approximately 1.85 inches and a thickness of approximately one quarter of an inch. According to embodiments, the length and width may be configured to be between approximately 5 times and approximately ten times the thickness of the magnet 402. In embodiments, the magnet housing 404 may include a generally rectangular shape. In embodiments, the length and width of the magnet housing 404 may be approximately equal, so that the magnet housing 404 has a generally square shape, which the inventors have discovered has the best drop test results.

FIG. 4C is a perspective view of the magnet assembly 400 depicted in FIGS. 4A and 4B, in accordance with embodiments of the subject matter disclosed herein; and FIG. 4D is a side view of the magnet assembly 400 depicted in FIGS. 4A-4C, in accordance with embodiments of the subject matter disclosed herein. As shown in FIGS. 4C and 4D, the magnet housing 404 may include one or more pry-off features 424 configured to facilitate gripping the magnet housing 404 and removing it from a surface to which it is adhered. As shown, a pry-off feature 424 may include, for example, a notch defined in the lower wall 406 and/or side wall 416 along one or more sides of the housing 404 and/or in one or more corners of the housing 404. In embodiments, the magnet housing 402 may include one pry-off feature 424, two pry-off features 424, three pry-off features 424, and/or the like. For example, in embodiments, the magnet housing 402 may include two pry-off features 424, in opposite corners (or on opposite sides) of the housing 402, at the ends of the longest dimension of the magnet housing 402, thereby increasing the amount of torque that a user can apply to the housing 402 to remove it from the surface to which it is adhered.

The illustrative magnet assembly 400 shown in FIGS. 4A-4D is not intended to suggest any limitation as to the scope of use or functionality of embodiments of the present disclosure. The illustrative magnet assembly 400 should not be interpreted as having any dependency or requirement related to any single component or combination of components illustrated therein. Additionally, various components depicted in FIGS. 4A-4D may be, in embodiments, integrated with various ones of the other components depicted therein (and/or components not illustrated), all of which are considered to be within the ambit of the subject matter disclosed herein.

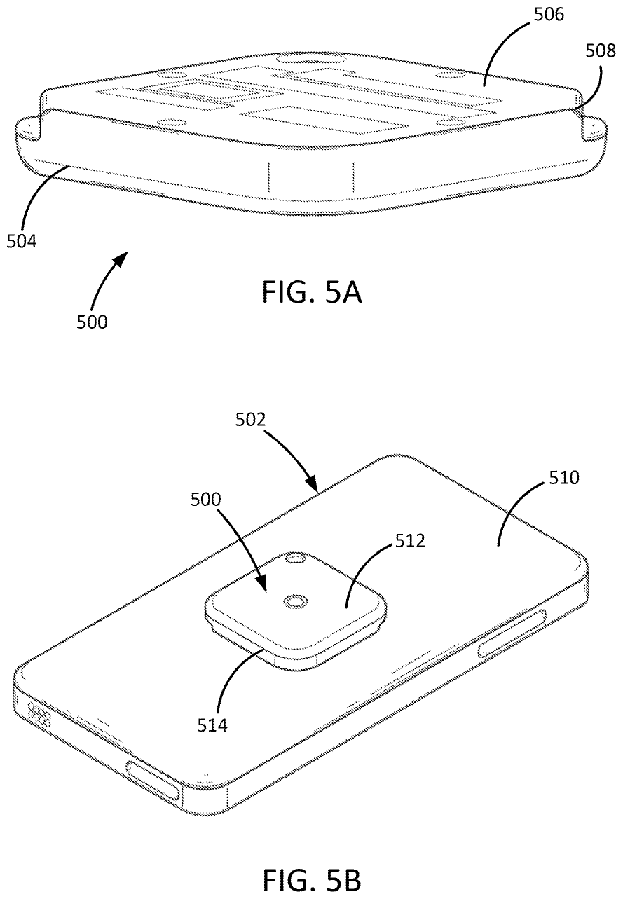

FIG. 5A is a lower perspective view of an illustrative magnet assembly 500, in accordance with embodiments of the subject matter disclosed herein; and FIG. 5B is a perspective view of the illustrative magnet assembly 500 depicted in FIG. 5A coupled to an illustrative mobile device 502, in accordance with embodiments of the subject matter disclosed herein. According to embodiments, the magnet assembly 500 may be, or be similar to, the magnet assembly 118 depicted in FIG. 1, the magnet assembly 300 depicted in FIGS. 3A-3D, and/or the magnet assembly 400 depicted in FIGS. 4A-4D. In embodiments, the mobile device 502 may be, or be similar to, the external device 106 depicted in FIG. 1.

As shown, the magnet assembly 500 includes a magnet housing 504 having a flat outer surface 506 of a lower wall 508. The flat outer surface 506 is configured to be coupled, via an adhesive, to an outer surface 510 of the mobile device 502. In embodiments, the adhesive may be, for example, a double sided adhesive tape that may be slightly smaller than the outer surface 506 of the lower wall 508 and may be configured according to any shape (e.g., circular, rectangular, etc.). As shown in FIG. 5B, coupling the magnet assembly 500 to the mobile device 502 in the manner illustrated exposes the outer surface 512 of the upper wall 514 so that the user can cause a change of state in an IMD simply by positioning the mobile device 502 near the subject's body, and facing the outer surface 510 toward the subject's body.

According to embodiments, the magnet assembly 500 may be configured according to any number of different shapes, sizes, and/or the like. According to embodiments, the magnet assembly 500 may be, include, or be included in any number of other types of devices configured to accessorize mobile devices. For example, the magnet assembly 500 may be, include, or be included in an accessory configured to facilitate coupling the mobile device 500 to a mount (e.g., a dashboard mount, a charging stand, etc.). In embodiments, the magnet assembly 500 may be configured to be, or be included in, a feature configured to be used as a handle for the mobile device 502 such as, for example, a PopSocket™, available from PopSockets LLC, of Boulder, Colorado.

The illustrative magnet assembly 500 and mobile device 502 shown in FIGS. 5A and 5B are not intended to suggest any limitation as to the scope of use or functionality of embodiments of the present disclosure. The illustrative magnet assembly 500 and mobile device 502 should not be interpreted as having any dependency or requirement related to any single component or combination of components illustrated therein. Additionally, various components depicted in FIGS. 5A and 5B may be, in embodiments, integrated with various ones of the other components depicted therein (and/or components not illustrated), all of which are considered to be within the ambit of the subject matter disclosed herein.

According to embodiments, the magnets described herein may be relatively strong (e.g., greater than or equal to approximately 1000 Gauss at the surface), as they need to be able to work with the magnetic sensors of the IMDs. Accordingly, it may be desirable to provide a packaging for shipping the magnet assemblies that facilitates reducing the magnetic field outside of the package.

Embodiments include a packaging assembly configured to facilitate transporting a magnet assembly. In embodiments, the packaging assembly may be a lightweight, stand-alone metal packaging configured to protect the surrounding environment (e.g., nearby devices, instruments, etc.) from the magnetic field produced by the magnet. In embodiments, the packaging assembly may be configured to maximize area inside in the direction of the magnetic field to aid in field dissipation, and/or to optimize dimensions and thickness of the container to provide desired shielding with minimal size and weight.

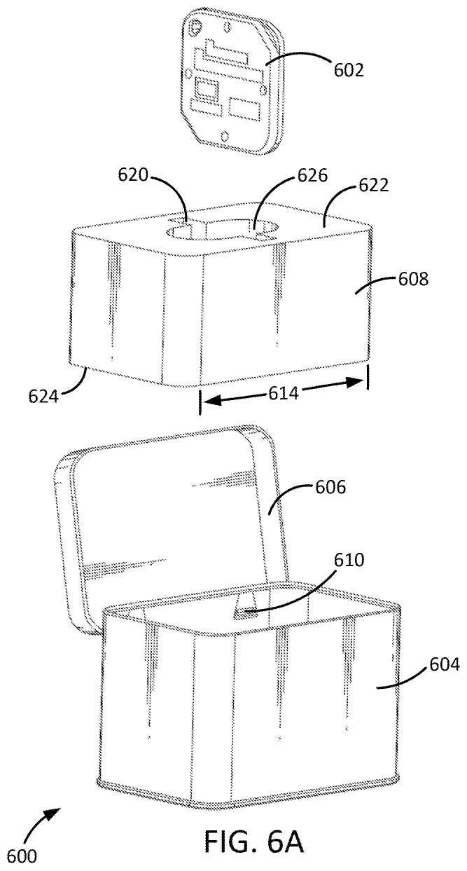

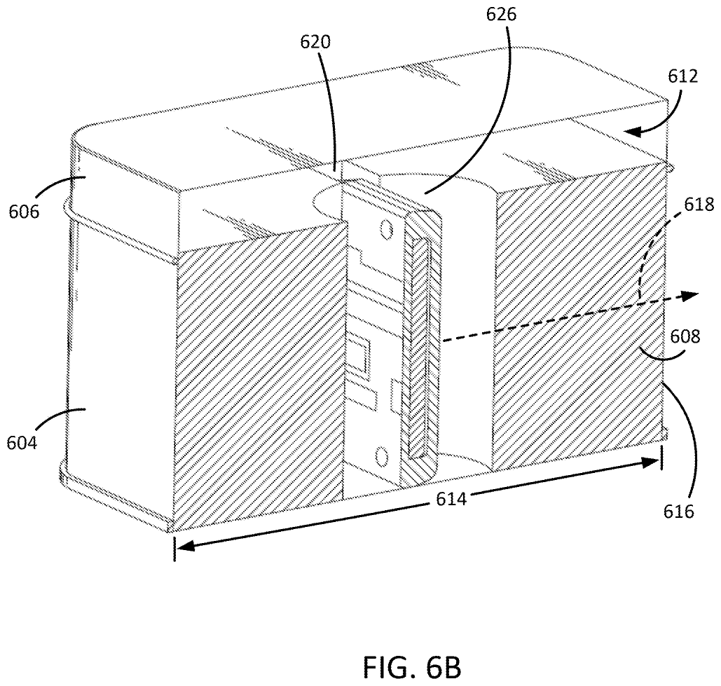

FIG. 6A is an exploded perspective view of an illustrative packaging assembly 600, in accordance with embodiments of the subject matter disclosed herein; and FIG. 6B is a cross-sectional perspective view of the illustrative packaging assembly 600, in accordance with embodiments of the subject matter disclosed herein. According to embodiments, the illustrative packaging assembly 600 may be used to transport a magnet assembly 602, and may be, in embodiments, provided with the magnet assembly as part of a medical device kit. The magnet assembly 602 may be, or be similar to, the magnet assembly 118 depicted in FIG. 1, the magnet assembly 300 depicted in FIGS. 3A-3D, the magnet assembly 400 depicted in FIGS. 4A-4D, and/or the magnet assembly 500 depicted in FIGS. 5A-5B.

As shown, the packaging assembly 600 includes a box 604 having a lid 606, and a foam insert 608 configured to be placed within the box 604. According to embodiments, the box may be made of any number of different materials including, for example, plastic, polymer, metal, and/or the like. For example, in embodiments, the box 604 may be made of tin-plated steel. In embodiments, the box 604 may be made of a solid material, multiple stacked materials, and/or the like. The lid 606 may be configured to be coupled to the box via hinges 610 so that the lid 606 is pivotable with respect to the box 604.

As shown in FIG. 6B, the foam insert 608 may be sized to fit snugly within the box 604 such that the foam insert 608 does not move within the box 604 during transport. The foam insert 608 and box 604 may be sized so that, when the foam insert 608 is disposed within the box 604, a space 612 may be provided for user manuals, adhesive tape (for coupling the magnet assembly 602 to an external device), and/or the like. According to embodiments, the foam insert 608 may be configured approximately as a rectangular cube, in which the length 614 (the longest dimension) is selected so as to provide a sufficient distance between the magnet assembly 602 and the outside surface 616 of the box 604 in the direction 618 of the magnetic field.

To center the magnet assembly 602 within the box 604, the foam insert 608 may include a slot 620 disposed at least partially through the foam insert 608. According to embodiments, the slot 620 may extend from an upper surface 622 of the foam insert 608, downward into the foam insert 608. In embodiments, the slot 620 may extend through the foam insert 608 to a lower surface 624 of the foam insert 608, while, in other embodiments, the slot 620 may terminate before reaching the lower surface 624. The slot 620 may be sized such that the magnet assembly 602 fits snugly within the slot 620 and is prevented from moving during transport. In embodiments, the foam insert 620 may further include an aperture 626 adjacent the slot 620 to facilitate insertion of a user's fingers or an instrument for removing the magnet assembly 602 from the foam insert 608. For example, as illustrated, the aperture 626 may include a curved cut-out on both sides of the slot 620. In other embodiments, the aperture 626 may include a cut-out on one side of the slot 620. The slot 620 may be configured to orient the magnet assembly 602 such that the direction 618 of the magnetic field is approximately parallel to the length 614 of the foam insert 608.

The illustrative packaging assembly 600 shown in FIGS. 6A and 6B is not intended to suggest any limitation as to the scope of use or functionality of embodiments of the present disclosure. The illustrative packaging assembly 600 should not be interpreted as having any dependency or requirement related to any single component or combination of components illustrated therein. Additionally, various components depicted in FIGS. 6A and 6B may be, in embodiments, integrated with various ones of the other components depicted therein (and/or components not illustrated), all of which are considered to be within the ambit of the subject matter disclosed herein. For example, in embodiments, the foam insert 608 may include a material configured to reduce magnetic flux.

Various modifications and additions can be made to the exemplary embodiments discussed without departing from the scope of the presently disclosed subject matter. For example, while the embodiments described above refer to particular features, the scope of this disclosure also includes embodiments having different combinations of features and embodiments that do not include all of the described features. Accordingly, the scope of the subject matter disclosed herein is intended to embrace all such alternatives, modifications, and variations as fall within the scope of the claims, together with all equivalents thereof.

We claim:

1. A magnet assembly configured to facilitate causing a state change of an implantable medical device (IMD), the IMD having a magnetic field sensor, the magnet assembly comprising:
a disc-shaped magnet; and
a magnet housing at least partially encasing the magnet.

2. The magnet assembly of claim 1, the magnet housing comprising a generally rectangular shape.

3. The magnet assembly of claim 2, wherein the magnet housing includes a length and a width, wherein the length is equal to the width so that the magnet housing has a generally square shape.

4. The magnet assembly of claim 1, wherein the magnet housing comprises an upper wall having an outer surface and an opposite inner surface, a lower wall having an outer surface and an opposite inner surface, and a side wall having an outer surface and an opposite inner surface.

5. The magnet assembly of claim 4, wherein the side wall of the magnet housing is tapered such that a length and/or width of the housing increases toward the lower wall.

6. The magnet assembly of claim 4, wherein the outer surface of the upper wall is approximately flat, thereby facilitating intuitive orientation of the magnet with respect to the IMD.

7. The magnet assembly of claim 4, wherein the outer surface of the lower wall is approximately flat to facilitate coupling the magnet housing to an external device.

8. The magnet assembly of claim 7, further comprising an adhesive configured to be disposed between the outer surface of the lower wall and an outer surface of the external device, wherein the adhesive is configured to couple the magnet housing to the external device.

9. The magnet assembly of claim 8, further comprising one or more pry-off features configured to facilitate gripping the magnet housing and removing it from the outer surface of the external device.

10. The magnet assembly of claim 9, wherein the one or more pry-off features comprise a notch defined in the lower wall of the magnet assembly.

11. The magnet assembly of claim 9, the one or more pry-off features comprising at least two pry-off features, in opposite corners of the magnet housing, at the ends of the longest dimension of the magnet housing.

12. The magnet assembly of claim 11, further comprising an aperture defined through the magnet housing.

13. A magnet assembly configured to facilitate causing a state change of an implantable medical device (IMD), the IMD having a magnetic field sensor, the magnet assembly comprising:
a disc-shaped magnet; and
a magnet housing at least partially encasing the magnet, wherein the magnet housing comprises an upper wall having an outer surface and an opposite inner surface, a lower wall having an outer surface and an opposite inner surface, and a side wall having an outer surface and an opposite inner surface, and wherein the side wall of the magnet housing is tapered such that a length and/or width of the housing increases toward the lower wall.

14. The magnet assembly of claim 13, further comprising an adhesive configured to be disposed between the outer surface of the lower wall and an outer surface of the external device, wherein the adhesive is configured to couple the magnet housing to the external device.

15. The magnet assembly of claim 14, further comprising one or more pry-off features configured to facilitate gripping the magnet housing and removing it from the outer surface of the external device.

16. The magnet assembly of claim 15, the one or more pry-off features comprising at least two pry-off features, in opposite corners of the magnet housing, at the ends of the longest dimension of the magnet housing.

17. The magnet assembly of claim 13, further comprising an aperture defined through the magnet housing.

18. A medical device kit, comprising:
a magnet assembly, the magnet assembly comprising a disc-shaped magnet and a magnet housing at least partially encasing the disc-shaped magnet; and
a packaging assembly comprising a box having a lid and a foam insert configured to be placed within the box, the foam insert comprising a slot defined therein for receiving the magnet assembly.

19. The medical device kit of claim 18, wherein the foam insert has a generally rectangular shape, wherein a length of the foam insert is greater than a width of the foam insert.

20. The medical device kit of claim 19, wherein the slot is configured to retain the magnet assembly in an orientation in which the direction of the magnetic field is approximately parallel to the length of the foam insert.
